(19) United States
(12) Patent Application Publication
(10) Pub. No.: US 2002/0044943 A1
(43) Pub. Date: Apr. 18, 2002

(54) MUC-1 as an immunosuppressive therapeutic agent for the treatment of inflammatory and autoimmune conditions

(76) Inventors: B. Michael Longenecker, Alberta (CA); Judith C. Hugh, Alberta (CA); Lyle H. Regimbald, Alberta (CA)

Correspondence Address: Bernhard D. Saxe, FOLEY & LARDNER, Washington Harbour, 3000 K Street, N.W., Suite 500, Washington, DC, 20007-5109, US

(21) Appl. No.: 09/927,389

(22) Filed: Aug. 13, 2001

Related U.S. Application Data
(62) Division of application No. 08/926,428, filed on Sep. 10, 1997.

(51) Int. Cl. A61K 39/00; A61K 31/715

(52) U.S. Cl. 424/185.1; 514/54

(57) ABSTRACT

The invention relates to novel methods of treatment and pharmaceutical compositions comprising the mucin MUC-1 and its derivatives. Preferred MUC-1 derivatives comprise multiple tandem repeats of the MUC-1 core sequence. The disclosed compositions and methods are particularly useful in treating autoimmune disorders, inflammatory disorders, organ transplant rejection and graft versus host disease.

### BACKGROUND OF THE INVENTION

Automimmune disorders represent a diverse collection of disorders, unrelated save for their common inflammatory etiology. Current treatments focus on this etiology and utilize a wide variety of medicaments, including non-steroidal antiinflammatories, corticosteroids, and even cytoablative agents. Unfortunately, neither the existing medicaments nor treatments which utilize them are wholly satisfactory. Likewise, similar dissatisfaction exists with respect to many inflammatory disorders, organ transplant rejection and graft-versus host disease. Thus, there exists a need for new medicaments and new methods of treatment for these disorders.

### SUMMARY OF THE INVENTION

It is, therefore, an object of the present invention to provide new methods for treating autoimmune disorders, inflammatory disorders, organ transplant rejection and graft versus host disease. According to this object, methods are provided which comprise administering a pharmaceutically effective amount of MUC-1, a MUC-1 derivative or a MUC-1 carbohydrate derivative to a patient in need of said treatment.

It is yet another object of the invention to provide novel pharmaceutical compositions of new and effective medicaments to implement methods for treating autoimmune disorders, inflammatory disorders, organ transplant rejection and graft versus host disease. According to this object of the invention pharmaceutical compositions are provided which comprise an amount of MUC-1, a MUC-1 derivative or a MUC-1 carbohydrate derivative effective for treating an autoimmune disorder, inflammatory disorder, organ transplant rejection or graft versus host disease, in combination with a pharmaceutically effective vehicle.

In preferred methods and compositions, the MUC-1 derivative comprises multiple tandem repeats of the MUC-1 core sequence.

### BRIEF DESCRIPTION OF THE DRAWINGS

FIG. 1 shows expression of MUC-1 at the surface of cultured murine (410.4) breast carcinoma cells transfected with the human MUC-1 gene as ascertained by flow cytometry using the antibody B27.29. Note the high MUC-1 expression (fluorescence intensity) of MUC-1Hi, which is approximately 2.5-fold higher than that of MUC-1Lo, whereas the wild-type 410.4 cell line does not express MUC-1.

FIG. 2 shows binding of MCF-7 cells to 4 hr stimulated HUVEc is inhibited most efficiently by anti-E selectin (Serotec 1.2B6) when endothelial monolayers are preincubated with monoclonal antibodies, and by anti-sLe^{x/a} (CSLEX & B67.4 sLe^{a}) when MCF-7 cells are pretreated with monoclonal antibodies (FIG. 2a). Binding of MCF-7 cells to 24 hr stimulated HUVEC is inhibited by anti-ICAM-1 (Serotec 84H10) (endothelial pretreatment) and by anti-MUC-1 (B27.29) (MCF-7 pretreatment) (FIG. 2b). Adhesion was assessed by a standard static endothelial adhesion assay described in Berry, et al. Br. J. Cancer 51:179 (1985). Results are expressed as a percentage of fluorescence signal from the total number of cells added to each well; each value represents the mean of three replicates +/− SD (error bars); ″ denotes wells coated with stimulated monolayers. The experiments shown are the best of three independent experiments of each type.

FIG. 3 shows high MUC-1 expressing transfectant (GZHi), which shows approximately four fold higher binding to 24 hr stimulated HUVEC than that of wild type (410.4), and approximately two fold higher binding than that of the low MUC-1 expresser (GZLO) (FIG. 3a) which is inhibitable by anti-MUC-1 (B27.29) and anti-ICAM-1(18E3D) (FIG. 3b). Adhesion was measured and the results are expressed as described in the description of FIG. 2. The experiments shown are the best of three independent experiments of each type.

FIG. 4a shows determination of serum MUC-1 levels by sandwich radioimmunoassay. FIG. 4b shows that lower serum MUC-1 levels correlate with longer survival time.

FIG. 5 shows high MUC-1 expressing cells (GZHi), which bind to immobilized recombinant soluble ICAM-1-Ig fusion protein and are inhibited by anti-MUC-1 (B27.29), anti-ICAM-1 (18E3D), and soluble MUC-1. Adhesion was measured as described in Takada, et al. Cancer Res. 53:354 (1993). The values shown are the means of three replicates +/− SD; ″ denotes ICAM-1 coated wells. The experiment shown is the best of four independent experiments.

FIG. 6 shows high MUC-1-expressing cells (MUC-1Hi) bind optimally to recombinant ICAM-1 (rhICAM-1) at 37° Ca. This adhesion mechanism is susceptible to almost complete inhibition at 4° C. Adhesion was measured and the results are expressed as described in the description to FIG. 3. The experiment shown is representative of three independent experiments. bars, standard deviation (SD).

FIG. 7 shows that addition of purified human MUC-1 to the in vitro human T cell culture inhibits T cell proliferative response against strong allogenic stimulus.

FIG. 8, panel a, shows the inhibition of T cell proliferation by the addition of 10 ug/ml of MUC-1 or a MUC-1 derivative, as compared to OSM or culture medium controls. Panels b and c show the abrogation of mucin-mediated T cell inhibition by anti CD28 Mab and IL-2, respectively.

FIG. 9 shows the direct relationship between the number of MUC-1 tandem core repeats and the inhibition of T cell proliferation.

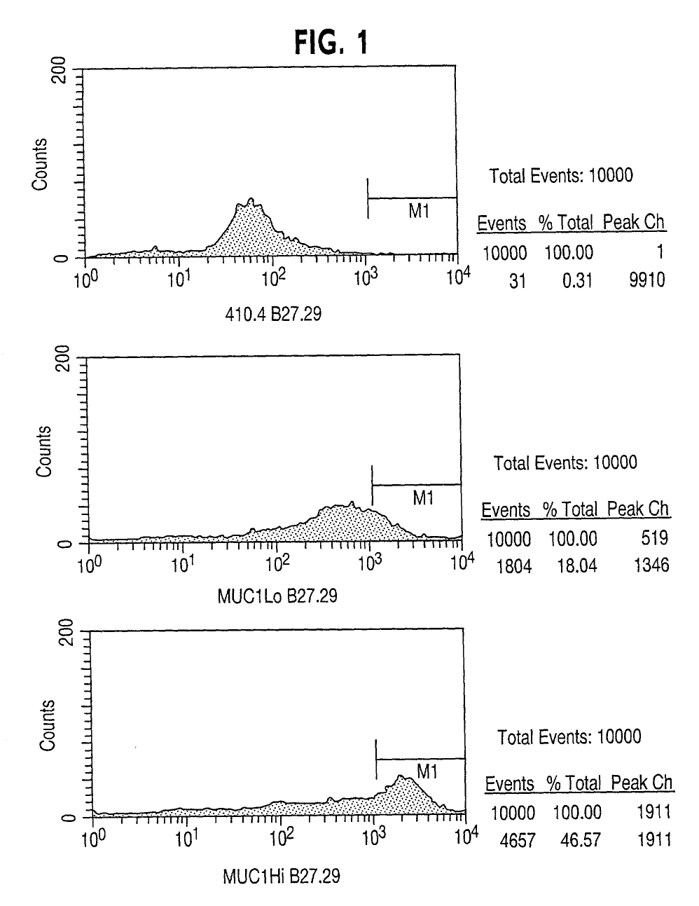

### DETAILED DESCRIPTION OF THE INVENTION

The present invention relates to the use of MUC-1 and its derivatives to treat inflammatory or autoimmune disorders, based on the ability of MUC-1 to bind to ICAM-1 and on evidence suggesting that MUC-1 has immunosuppressive effects in tumor models. The present invention also relates to use of MUC-1 and its derivatives to suppress or prevent transplant rejection and graft versus host reactions. It is known that both inflammatory and autoimmune disorders are associated with a hyperreactive, or overreactive, immune response. Thus, MUC-1 and its derivatives may be employed as immunosuppressive agents to treat these disorders by suppressing the overreactive immune response.

Background

Mucins are a family of glycoproteins, greater than 200 kDa. Some mucins, such as MUC-1, are membrane bound molecules with an extended extracellular domain composed of tandem repeats of amino acid (aa) sequences which contain numerous potential O-glycosylation sites. Devine, et al. BioEssays 14:619 (1992).

Numerous clinical studies have suggested that mucinous tumor antigens, both expressed on the cell surface of tumor cells and shed from the surface of tumor-cells, are associated with a poor prognosis of a variety of cancer types. See, for example Itzkowitz, et al. Cancer 66:1960 (1990).

Over ninety percent of breast cancers show an increased expression of a membrane bound molecule, MUC-1 (Episialin, Epithelial Membrane Antigen, Polymorphic Epithelial Mucin, Human Milk Fat Globule Membrane antigen etc.). Berry, et al. Br. J. Cancer 51:179 (1985). MUC-1 is a well-characterized tumor cell surface mucin which is shed into the serum and carries repeating sialyl-Tn epitopes. See Cancer Research 47:5476 (1987) and Reddish, et al. Glycoconjugate Journal 13:1 (1996). MUC-1 is a highly glycosylated mucin type glycoprotein present on the luminal surface of most glandular epithelia and is often found increased over the entire surface of many carcinoma cells. This mucin extends far above the cell surface making it easily available for interactions with other cells. MUC-1 is expressed in normal breast and in approximately 90% of breast cancers. MUC-1 is present as both a transmembrane form as well as in a secreted form. It is not known if both show similar patterns of glycosylation.

Mucins are known to lubricate mucosal surfaces providing protection, and cell surface mucins are thought to participate in cell-cell interactions. See Samuel and Longenecker In Vaccine Design: The Subunit and Adjuvant Approach, Powell, et al., eds. (Plenum Press, N.Y. pp. 875-890 (1995)). Some investigators support an anti-adhesive function for MUC-1 through steric hindrance and negatively charged O-linked sialic acid residues. Ligtenberg, et al. Cancer Res. 52:2318 (1992).

The extracellular and cytoplasmic domains can support intercellular adhesion. The extracellular domain is composed of 30 to 90 tandem repeats of a highly glycosylated twenty aa sequence. The presence of numerous highly glycosylated tandem repeats allows MUC-1 to extend 200-500 nm above the cell surface far beyond the surrounding 10-30 nm glycocalyx. This places it in an ideal position to enhance adhesion with those cells possessing the appropriate receptor(s). Sialylated Lewis^{x/a} residues, which are also carried by MUC-1, mediate the adhesion of malignant colonic cells to E-Selectin. Sawada, et al. Int. J. Cancer 57:901 (1994), Zhang, et al. Int. J. Cancer 59:823 (1994). The 69 aa cytoplasmic domain of MUC-1 is linked to microtubules of the cytoskeleton, which is similar to the structure of other adhesion moieties. This interaction with the submembraneous actin cytoskeletal filaments is thought to account for the preferential expression of MUC-1 on free or unattached membrane surfaces. Parry, et al. Exp. Cell Res. 188:302 (1990).

The presence of membrane bound MUC-1 could facilitate tumor cell interactions with stimulated endothelial cells during the process of blood borne metastases. Several carcinoma cell lines adhere in vitro to activated endothelial cells using molecules similar to those employed by normal extravasating inflammatory cells. Takada, supra, Carlos, et al. Blood 84:2068 (1994). In this process, high affinity interactions between endothelial cell immunoglobulin superfamily members such as ICAM-1 or VCAM are thought to involve selective interactions with members of the Integrin family. Carlos, supra.

ICAM-1 (Intracellular Adhesion Molecule) is expressed on B cells and T-cells and antigen presenting cells (APCs) where it participates in cell-cell interactions important for the induction of an immune response. It is also present in high levels on endothelial cells. ICAM-1 (CD 54) is found on most cells of the immune system, particularly on cytokine-activated cells, of both hemolytic and non-hemolytic origin fibroblasts and keratinocytes.

Recently CD43, a highly glycosylated mucin type glycoprotein with no structural resemblance to the beta 2 integrins (CD 11a/b with CD18) has been reported to bind ICAM-1. Rosenstein, et al. Nature 354:233 (1991). This suggests that there is pliancy in the receptor-ligand recognition so that members of different receptor classes could interact. Thus, it is possible that MUC-1 mediates adverse effects through an interaction with adhesion molecules, such as ICAM-l.

Support for an ICAM-1/MUC-1 interaction can be drawn from studies of cytotoxic T lymphocytes and MUC-1 transfected cells. These interactions are shown to be non-MHC restricted and are ICAM-1 dependent. van de Weil-van Kemmenade, et al. Immunology 151:767 (1993). They also are inhibited by SM3, an antibody against the aa sequence DTRP of the MUC-1 tandem repeat. Jerome, et al. Immunology 151:1654 (1993). This suggests that the ICAM-1 binding site on MUC-1 lies within the peptide core, which is uniquely exposed by the cancer-associated underglycosylation of MUC-1.

In breast cancer, the amount of membrane-bound MUC-1 is increased. The mucin is also altered with fewer carbohydrate residues, thereby exposing usually cryptic epitopes on the protein core and interior carbohydrates. This cancer-associated configuration forms the basis for anticancer immunotherapy. Longenecker, et al. The Immunologist 1:89 (1993), Agrawal, et al. Cancer Res. 55:2257 (1995). In advanced breast cancer, MUC-1 is shed from tumor cells and is thus elevated in the serum, where it correlates with an unfavorable prognosis and possibly immunosuppression through the induction of T-cell anergy. Reddish et al. Cancer Immunol Immunother. 42:303-09 (1996).

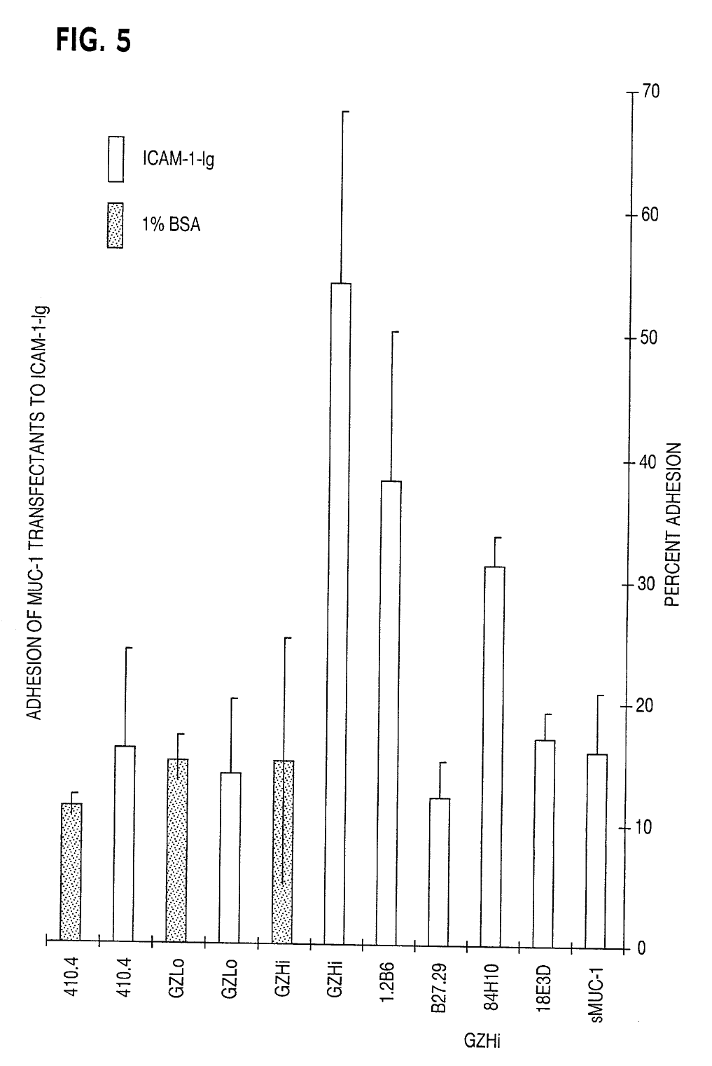

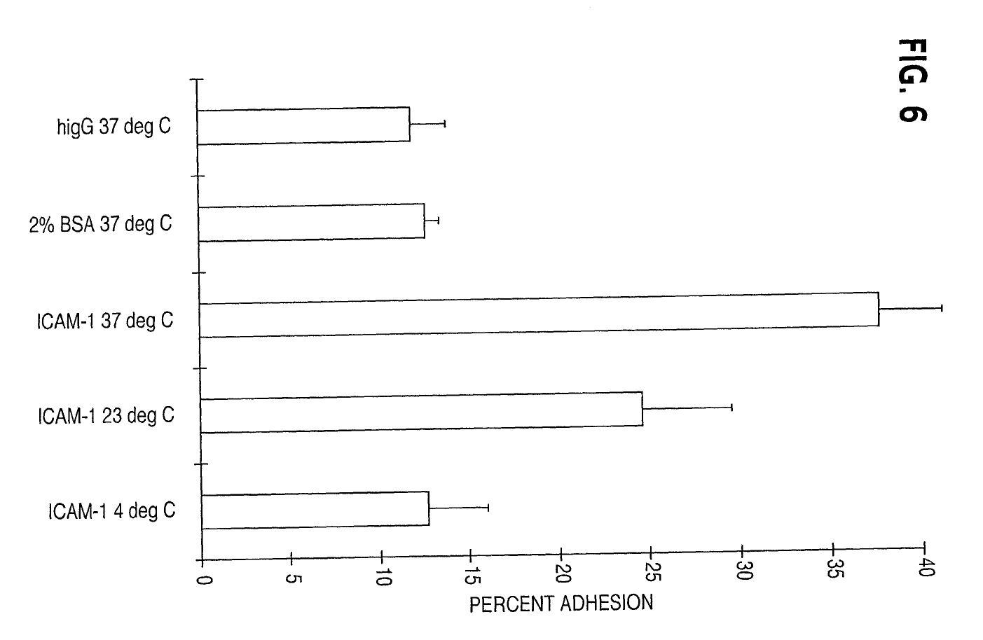

Because soluble MUC-1 can competitively inhibit adhesive interactions of MUC-1-positive cells with ICAM-1 (FIG. 5), it is possible that serum MUC-1 is inducing the anergic state by occupying ICAM-1 receptors on cytotoxic T-cells (CTLs). By occupying peritumoral endothelial cell ICAM-1 receptors, serum MUC-1 could also inhibit adhesive interactions of migrating cells with the endothelium and thus (a) cause decreased recruitment of inflammatory cells to the tumor site and (b) facilitate tumor cell escape and metastasis from the primary mass.

Preparations of MUC-1 and MUC-1 Derivatives

The MUC-1 used in the compositions and methods of this invention may be purified from sources such as cancer cell lines secreting MUC-1 and pleural effusions or ascites fluid from cancer patients. See Example 8. The MUC-1 used in the methods of this invention may also be obtained by recombinant DNA techniques that are well known to those of skill in the art. See, for example Gendler, et al., J. Biol Chem. 265:15286 (1990).

As used herein a “MUC-1 derivative” is used to refer to a peptide that is structurally and/or functionally related to MUC-1. Such derivatives may retain some or all of the functional characteristics of MUC-1, in particular, the immunosuppressive function of MUC-1. The immunosuppressive function easily may be measured using the assays set for below in the Examples.

A MUC-1 derivative may be partially deglycosylated or completely unglycosylated MUC-1 protein. Deglycosylation of MUC-1 protein can be carried out using techniques that are well known to the skilled artisan.

A MUC-1 derivative may be a fragment of the MUC-1 protein. Such fragments may be glycosylated or unglycosylated. In accordance with the present invention, fragments within the invention can be obtained from purified MUC-1 or MUC-1 produced by recombinant DNA methodology by methods that include digestion with enzymes such as pepsin or papain. Alternatively, MUC-1 fragments encompassed by the present invention can be synthesized using an automated peptide synthesizer such as those supplied commercially by Applied Biosystems, Multiple Peptide Systems and others, or they may be produced manually, using techniques well known in the art. See Geysen et al., J. Immunol. Methods 102:259 (1978).

MUC-1 derivatives also include glycosylated or non-glycosylated synthetic peptides. In addition, MUC-1 derivatives within the present invention include proteolytic cleavage-resistant MUC-1 fragments or MUC-1 fragments containing one or more non-natural amino acids, such as D-amino acids.

In another embodiment, the MUC-1 derivative would include the extracellular tandem repeat region of MUC-1, which includes repeats of the amino acid sequence DTRP (Asp-Thr-Arg-Pro). Preferrably these tandem repeats include the sequence SAPDTRP (Ser-Ala-Pro-Asp-Thr-Arg-Pro).

Some preferred MUC-1 derivatives comprise at least one peptide core repeat of the MUC-1 mucin. A MUC-1 peptide core repeat in the native MUC-1 molecule comprises the 20 amino acid sequence PDTRPAPGSTAPPAHGVTSA (Pro-Asp-Arg-Thr-Pro-Ala-Pro-Gly-Ser-Thr-Ala-Pro-Pro-Ala-His-Gly-Val-Thr-Ser-Ala). Useful synthetic derivatives include permutations of this sequence, for example, GVTSAPDTRPAPGSTAPPAH, where the repeat merely begins with GVTS rather than PDTR. Other, similar permutations are also possible.

Moreover, one or more amino acids of the core sequence may be altered, preferrably in a conservative manner known in the art, such that the requisite immunosuppressive activity is maintained. Other MUC-1 derivatives comprise at least one truncated peptide core repeat of the MUC-1 mucin, for example, GVTSAPDTRPAPGSTA. Of course any combination of core sequence permutation, alteration or truncation may be linked together to form multiple, and especially tandem multiple repeats.

Data presented below in Examples 8 and 9 demonstrate that fewer than 1 core repeat fails to exhibit the MUC-1 immunosuppressive function. But, where more than 1 repeat is present, it is observed that the degree of immunosuppression increases linearly with the number of repeats present. Thus, although an upper limit to the number of repeats that effectively can be employed is likely, MUC-1 derivatives containing very large numbers of repeats are contemplated. For example, the native MUC-1 contains 60-100 of such repeats.

Some embodiments contemplate from about 2 to about 100 core repeats. However, more preferred MUC-1 derivatives comprise from 2-20 repeats of the MUC-1 core. Most preferred MUC-1 derivatives comprise from 3-6 repeats of the MUC-1 core and the repeats preferably are arranged in tandem.

Of course, as described above, these preferred MUC-1 derivatives may be glycosylated or partially glycolysated according to methods known in the art. Moreover, it is contemplated that MUC-1 and MUC-1 derivatives can be modified with large molecular weight polymers, such as polyethylene glycols.

Also illustrative of an MUC-1 derivative within the present invention is a non-peptide “mimetic,” i.e., a compound that mimics one or more functional characteristics of the MUC-1 protein. Mimetics are generally water-soluble, resistant to proteolysis, and non-immunogenic. Conformationally restricted, cyclic organic peptides which mimic MUC-1 can be produced in accordance with known methods described, for example, by Saragovi, et al., Science 253:792 (1991).

“MUC-1 carbohydrate derivatives” are also contemplated. Such a derivative, as used herein, refers to a glycopeptide which retains at least one functional characteristic of MUC-1, such as immunosuppression. Such a carbohydrate derivative may include all or part of the carbohydrate that is attached to the MUC-1 protein. Mimetics that mimic at least one property of MUC-1 carbohydrate may also be used.

Designing Other MUC-1 Derivatives

In another embodiment, MUC-1 derivatives may be designed to block ICAM-1-mediated cell interactions to effect immunosuppression. In antigen specific T-cell responses, T-cells interact via T-cell receptors (TCRs), with antigen-presenting cells (APCs). In addition to interaction of TCR with MHC complex molecules during antigen presentation to T-cells, a number of other interactions between APCs and TCR are required, using various accessory molecules (co-receptors). Interaction of accessory molecules is important in a T-cell response because these interactions (1) increase the avidity of interaction between the APC and the T-cell and (2) induce various intracellular signal pathways in the T-cells.

Interaction of ICAM-1 with its ligand is important in conjunction with TCR ligation to produce an efficient T-cell stimulatory signal. However, when the ICAM-1 interaction with its ligand and cross-linking occurs in the absence of TCR ligation, the T-cells become anergic (non-responsive). Therefore, MUC-1 or its derivatives can be used as immunosuppressive agents: when MUC-1 or its derivatives interacts with and cross-links ICAM-1 molecules in the absence of an antigenic stimulus, this results in nonresponsiveness (immunosuppression) of the T-cells.

Thus, in this embodiment, MUC-1 derivatives may be designed to block MUC-1/ICAM-1 interactions which will reduce the ICAM-1-mediated cellular immune response. In one embodiment, such blocking MUC-1 derivatives may be designed by using MUC-1 derivative binding to recombinant human (rh) ICAM-1-immunoglobulin (Ig) fusion protein. In this method, the fusion protein is immobilized in the solid phase. Libraries of synthetic MUC-1 peptides, glycopeptides and glycoconjugates based on the MUC-1 tandem repeat can be screened for blocking activity. Those compounds that partially or completely block MUC-1 binding to ICAM-1 may be screened for therapeutic effectiveness.

The multiple copies of the MUC-1 tandem repeat contained in the MUC-1 protein may be able to cross-link several ICAM-1 molecules, leading to immune suppression. The “libraries” of synthetic peptides containing a single copy of the tandem repeat should not produce this effect. Similarly, those peptides that contain less than an entire tandem repeat should not produce this effect. The data presented below in Examples 8 and 9 support this view.

Once potential immunosuppressive MUC-1 derivatives have been identified using the ICAM-1/MUC-1 binding assay, the derivatives can be further evaluated using a T-cell based immune suppression system. Such a system determines the ability of a derivative to prevent in vitro T-cell induction.

Because MUC-1 interacts with ICAM-1, and is expected to react with other adhesion molecules such as ICAM-3, various MUC-1 derivatives should be useful as drugs that block the MUC-1/adhesion molecule interaction. Such adhesion inhibitors could be used to treat cancer patients who are immunosuppressed by MUC-1 mucin.

Pharmaceutical Formulations

The pharmaceutical compositions of the invention generally contain a pharmaceutically effective amount of MUC-1, a MUC-1 derivative or a MUC-1 carbohydrate derivative. Preferably, MUC-1, a MUC-1 derivative or a MUC-1 carbohydrate derivative is admixed with a pharmaceutically effective vehicle (excipient).

A suitable formulation will depend on the nature of the disorder to be treated, the nature of the medicament chosen, the route of administration desired and the judgment of the attending physician. Suitable formulations and pharmaceutically effective vehicles, can be found, for example, in REMINGTON'S PHARMACEUTICAL SCIENCES, chapters 83-92, pages 1519-1714 (Mack Publishing Company 1990) (Remington's), which are hereby incorporated by reference.

Methods of the Invention

MUC-1, a MUC-1 derivative or a MUC-1 carbohydrate derivative may be used to treat autoimmune disorders and inflammatory disorders. MUC-1 may also be used to prevent or suppress organ transplantation rejection and graft versus host disease in bone marrow transplantation. Thus, the methods of the invention typically comprise administering a pharmaceutically effective amount of MUC-1, a MUC-1 derivative or a MUC-1 carbohydrate derivative to a patient in need of treatment. The patient may be a human or non-human animal.

As used herein, an “inflammatory disorder” refers to any of the many inflammatory disorders that are well known to those of skill in the art. These disorders include, but are not limited to, the following disorders: inflammatory arthritis such as rheumatoid arthritis, psoriasis, allergies such as allergic contact dermatitis, and ankylosing spondylitis.

As used herein, an “autoimmune disorder” refers to any of the many autoimmune disorders that are well known to those of skill in the art. These disorders include, but are not limited to, the following disorders: myasthenia gravis, systemic lupus erythematosus, polyarteritis nodosa, Goodpastures syndrome, isopathic thrombocytopenic purpura, autoimmune hemolytic anemia, Grave's disease, rheumatic fever, pernicious anemia, insulin-resistant diabetes mellitus, bullous pemphigold, pemphigus vulgaris, viral myocarditis (Cocksakie B virus response), autoimmune thyroiditis (Hashimoto's disease), male infertility (autoimmune), sarcoidosis, allergic encephalomyelitis, multiple sclerosis, Sjorgens disease, Reiter's disease, Celiac disease, sympathetic ophthalmia, and primary biliary cirrhosis.

Intracapsular, intravenous, intrathecal, and intraperitoneal routes of administration of MUC-1 and its derivatives may be employed. The skilled artisan will recognize that the route of administration will vary depending on the disorder to be treated. For example, intracapsular administration may be used when treating arthritis. Injection into the hepatic portal vein may be employed when treating inflammatory hepatitis. Intra-organ injection of the thyroid may be used when treating thyroiditis.

Either intravenous or intraperitoneal administration may be used when treating autoimmune diseases of the gastrointestinal tract, such as pancreatitis or colitis. Intrathecal administration may be appropriate when treating autoimmune encephalitis.

Intravenous or intra-organ injections may be employed to prevent or suppress transplant rejections, such as kidney transplants.

The term “treating” in its various grammatical forms in relation to the present invention refers to preventing, curing, reversing, attenuating, alleviating, minimizing, suppressing or halting the deleterious effects of a disease state, disease progression, disease causative agent (e.g., bacteria or viruses) or other abnormal condition.

Determining a pharmaceutically effective amount of MUC-1, a MUC-1 derivative or a MUC-1 carbohydrate derivative is well within the purview of the skilled clinician and largely will depend on the exact identity of the inventive compound, particular patient characteristics, route of administration and the nature of the disorder being treated. General guidance can be found, for example, in the publications of the International Conference on Harmonisation and in REMINGTON'S PHARMACEUTICAL SCIENCES, chapters 27 and 28, pp. 484-528 (Mack Publishing Company 1990).

Determining a pharmaceutically effective amount specifically will depend on such factors as toxicity and efficacy of the medicament. Toxicity may be determined using methods well known in the art and found in the foregoing references. Efficacy may be determined utilizing the same guidance in conjunction with the methods described below, for example, in Examples 8 and 9. A pharmaceutically effective amount, therefore, is an amount that is deemed by the clinician to be toxicologically tolerable, yet efficacious.

The foregoing discussion and following examples are presented merely for illustrative purposes and are not meant to be limiting. Thus, one skilled in the art will readily recognize additional embodiments within the scope of the invention that are not specifically exemplified.

### EXAMPLES

### Example 1

This example shows that mice bearing MUC-1 transfected tumor cells (which secrete MUC-1 into the serum) who develop the highest serum MUC-1 levels following tumor transplantation are the mice showing the shortest survival time compared to mice who had non-detectable levels of serum MUC-1 following tumor transplantation.

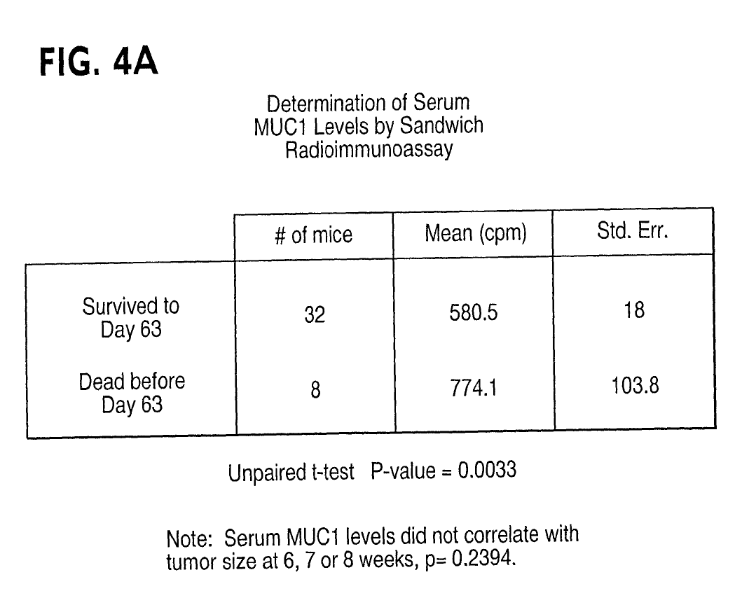

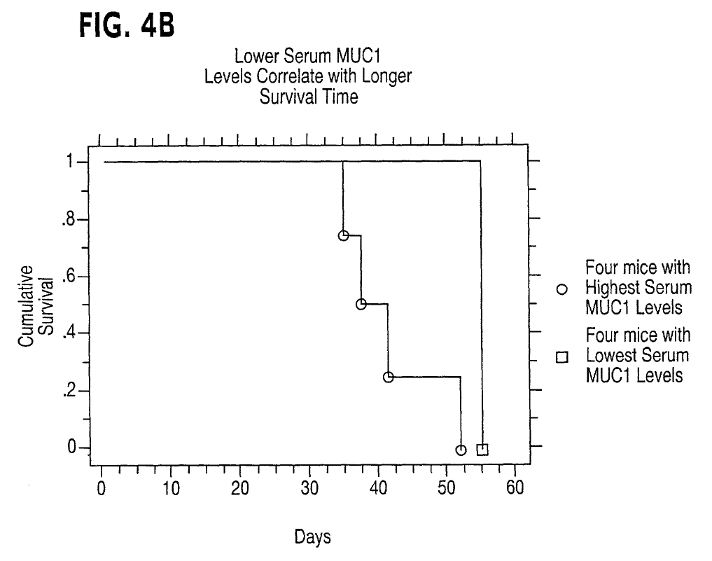

MUC-1Hi cells were transplanted subcutaneously and tumors developed in over 90% of the mice within 6-8 weeks. Mice were followed for survival and serum MUC-1 levels were measured at various times following tumor transplant. FIG. 4a shows that the 8 mice that died before day 63 had higher serum MUC-1 levels than those that survived to day 63. FIG. 4b shows that of the 8 mice who died before day 63, there was a significant direct association between MUC-1 level and survival time.

### Example 2

This example shows that intravenous injection of MUC-1 mucin made mice more susceptible to the tumor transplant of human MUC-1 transfected tumor cells. The procedures employed in this example are similar to those described in Fung, et al. Cancer Research 51:1170-1176 (1991), which is hereby incorporated by reference. However, in the present example, the human MUC-1 system was used instead of the epiglycanin system.

CB6 mice were injected intravenously with MAb B27.29 affinity purified MUC-1 mucin (20 micrograms each treatment or approximately 400 units) prior to (day-2) and after (days +2 and +6) an attempted tumor challenge. Affinity purified mucin had been prepared by affinity adsorption on a B 27.29 CnBr-Sepharose matrix from a pleural effusion fluid obtained from a patient with adenocarcinoma of the ovaries. MUC-1 mucin treated and control mice were challenged with 1×10⁶ GZ-Hi tumor cells injected into the subcutaneous space along the flank. Mice were observed by palpation and visual inspection at the injection site for the occurrence of tumors over a period of 8 weeks.

Results:

| Experimental Group | % Tumor Take |
|---|---|
| Controls | 60% |
| MUC-1 pretreated | 100% |

### Example 3

This example shows that adding purified human MUC-1 mucin to human T-cell cultures strongly inhibits T-cell proliferation against a strong allo-antigenic stimulus in vitro.

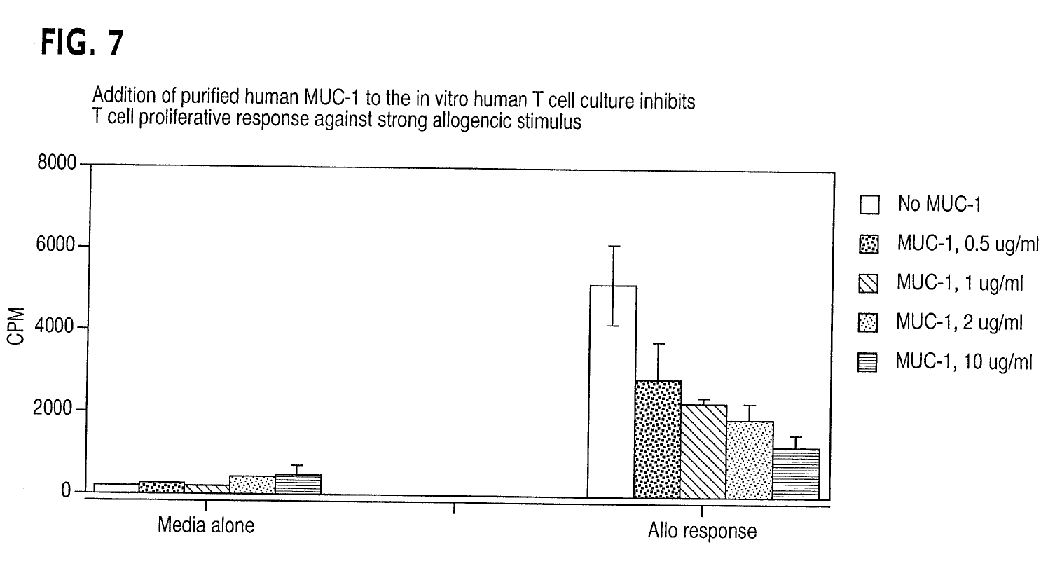

The mixed lymphocyte reaction is conducted by mixing the lymphocytes of HLA disparate individuals in in vitro tissue cultures. The “responder population” in this experiment is purified T-cells from one population, while the “stimulator” population in this experiment is the adherent antigen presenting cells obtained from an HLA mismatched individual donor. The two cell populations were mixed and cultured either in the presence or absence of various doses of B27.29 affinity purified MUC-1 mucin that was purified from a pleural effusion fluid. The results of this experiment are presented in FIG. 7.

### Example 4

This example shows that MUC-1 mucin binds to ICAM-1. Tumor cells that are transfected with human MUC-1 bound to ICAM-1 on endothelial cells. MUC-1 appears to be a ligand for Intercellular Adhesion Molecule (ICAM-1). Antibodies to ICAM-1 and MUC-1 inhibited adhesion of MUC-1 positive cells to human umbilical vein endothelial cell (HUVEC) monolayers in a manner directly related to the level of MUC-1 expression. Similar antibody inhibition of adhesion of MUC-1 positive cells was found using ICAM-1 transfected cells and immobilized recombinant human ICAM-1-Ig fusion protein. These results suggest that ICAM-1 binds to the peptide core of the tandem repeat sequence of the MUC-1 molecule thereby implicating MUC-1 in breast cancer metastases and immune suppression.

MUC-1 preferentially localizes to free membrane surfaces including a peripheral ensheathing pattern of staining on the surface of intravascular tumor emboli. Tumor emboli stained for MUC-1 using the DAB immunoperoxidase technique showed intense peripheral staining in contrast to solid tumor nests which show a diffuse cytoplasmic staining pattern. There was uneven staining along the endothelial surface, which probably represents shed antigen.

Several mucins have recently been described as vascular ligands. Berg, et al. Nature 366:695 (1993). Thus, the staining pattern of MUC-1 suggested that MUC-1 may be involved in endothelial cell adhesion.

Tumor Cell—Endothelial Cell Adhesion Assay:

Endothelial cell monolayers were grown in 24 well tissue culture plates and stimulated with 20 U/mL TNFα+20 U/mL IL-1β(Phariningen) for 4 or 24 hr. To these plates BCECF AM ester (Molecular Probes) fluorescent dye labeled tumor cells (1.5×10⁵/500 uL/well) were added and incubated for 25 min. at 37 C. Non-specifically adherent cells were removed by vigorous agitation (shaker at 175 rpm) and peripheral aspiration. Remaining adherent cells were then lysed with detergent (NP-40) for 30 min. and the dye signal was quantitated using a SPEX fluorimeter and compared to the signal from the total number of cells added to each well.

Tumor Cell—Immobilized Icam-1 Adhesion Assay:

96 well tissue culture plates were coated with 50 uL of a solution with 20 ug/mL recombinant soluble ICAM-1 in PBS for 1 hr at room temperature. Wells were then blocked with 1% BSA for 2 hr at 37 C. and washed four times with PBS. Antibodies (10 ug/mL) (anti-ICAM-1 164B, 18E3D, 84H10, anti-E selectin 1.2B6) and soluble MUC-1 (10 ug/mL) were added to the wells for 90 min. at room temperature. Wells were then washed again before addition of cells. BCECF labeled tumor cells were pretreated +/− 20 ug/mL of anti-MUC-1 (B27.29, or DF3P) and were then added to the appropriate wells for 40 min. at 37 C. Wells were then washed and percent adhesion was then determined as described in the tumor cell—endothelial cell adhesion assay.

Results:

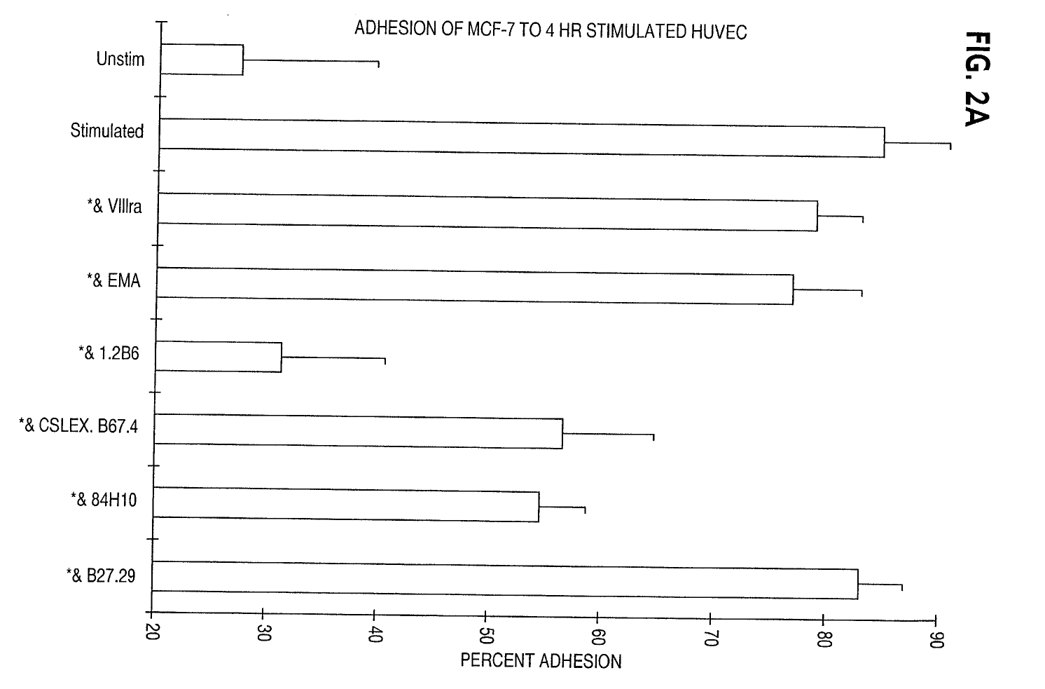

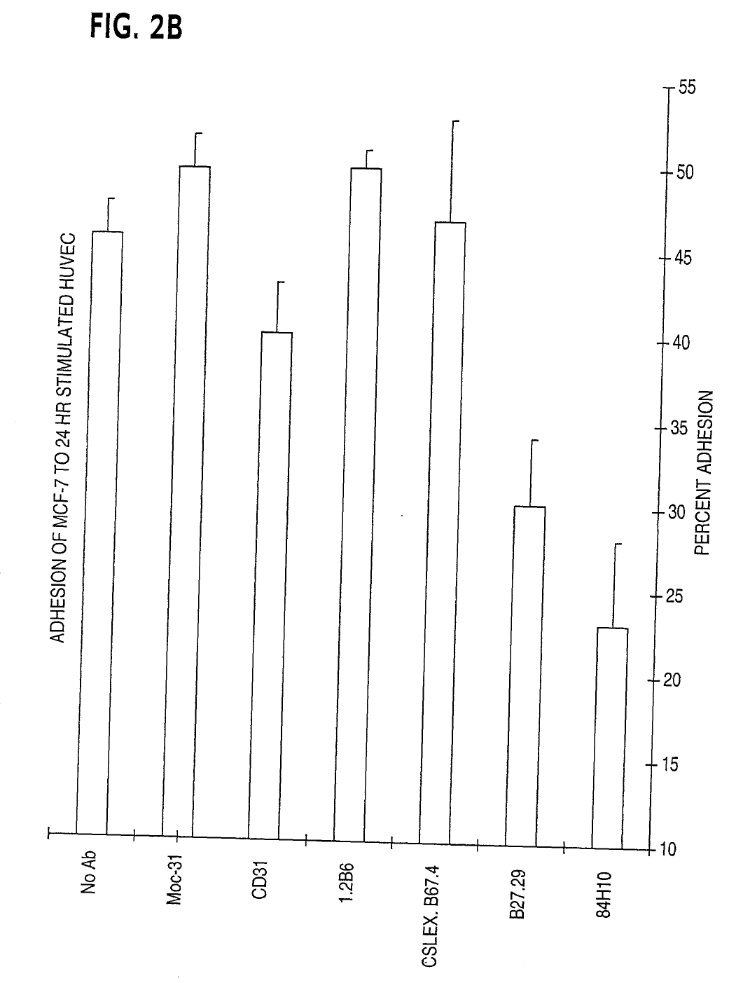

A series of monoclonal antibodies were screened for their ability to inhibit the binding of a human breast carcinoma cell line (MCF-7) to human umbilical vein endothelial cell (HUVEC) monolayers which had been stimulated with IL-IB and TNF-α. Consistent with other studies of epithelial cancer cell lines, following four hour cytokine stimulation of the HUVECS, antibodies to E selectin or its ligands (SLe^{x/a}) showed the greatest inhibition of adhesion of the MCF-7's to the HUVECs (FIG. 2a). Antibody to ICAM-1 (84H10) also showed inhibition but much less than that obtained by anti-E selectin (1.2B6). An antibody to MUC-1 (B27.29), had no effect. The adhesion molecule profile of cytokine stimulated HUVEC's varies over time. See Iwai, et al., Int'l Journal of Cancer 54:972 (1993). E-Selectin is expressed early with a maximum peak at 4 hours and is gradually lost by 22 hours. ICAM-1 is also expressed at 4 hours but it remains highly expressed after 22 hours. Thornhill, et al. Scand. J. Immunology 38:279 (1993). Therefore, the adhesion assay was repeated after prolonging endothelial stimulation to 24 hours. Under these conditions antibodies to E selectin and sLe^{x/a} now showed very little inhibition (FIG. 2b). Consistent with the adhesion molecule profile at 24 hours, antibodies to ICAM-1 showed substantial inhibition of tumor cell-HUVEC adhesion. Interestingly, the MUC-1 antibody (B27.29) also showed quite potent inhibition of the MCF-7 cells to the HUVEC's (FIG. 2b). This suggested that MUC-1 could be an additional mucin type ligand for ICAM-1.

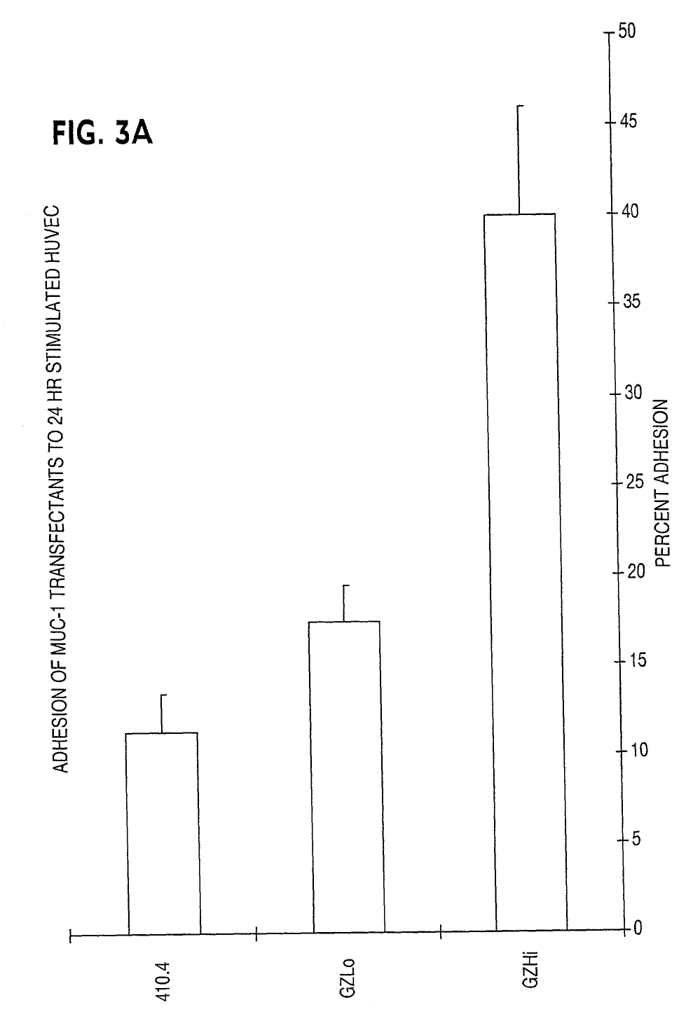

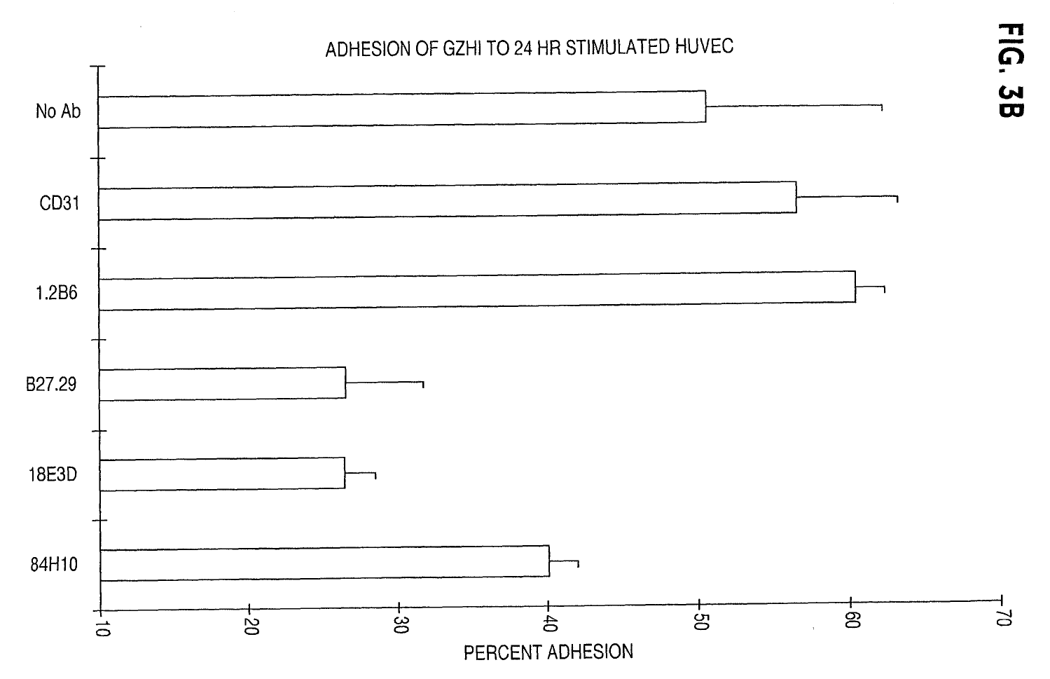

To isolate MUC-1 as the relevant adhesion molecule, the 24 hour adhesion assay was repeated using the parental murine 410.4 mammary adenocarcinoma cells as well as two derivative cell lines transfected with the human MUC-1 gene but showing either low (GZLO) or high (GZHi) expression of MUC-1. The level of MUC-1 expression was quantitated by concurrent flow cytometric analysis as well as by immunohistochemistry. FIG. 3a shows an increase in adhesion with increasing MUC-1 expression with GZHi showing a two fold increase in adhesion over GZLO and a four fold increase over the wild type (410.4). This adhesion was inhibited by antibodies to MUC-1 (B27.29) and ICAM-1 (18E3D) while anti-E selectin and control antibodies (CD31) showed no effect (FIG. 3b).

To exclude the possibility that adhesion was being mediated by endothelial molecules other than ICAM-1, the adhesion of the MUC-1 transfectants was studied using immobilized recombinant human ICAM-1-Ig fusion protein. Adhesion was compared to Bovine Serum Albumin and Collagen type I as controls. 410.4 and GZLO cells displayed similarly low binding to 1% BSA and ICAM-1 coated wells. GZHi however, showed approximately 3.5 fold higher binding to ICAM-1 than did 410.4 and GZLO, while its binding to 1% BSA was comparable to that of 410.4 and GZLO (FIG. 5). In addition, anti-MUC-1 (B27.29) and anti-ICAM-l (18E3D) antibodies successfully abrogated the increased adhesion of GZHi to ICAM-1. These antibodies did not, however, inhibit adhesion of high MUC-1 expressing cells to a collagen type I control in subsequent experiments. Pretreatment of the ICAM-1 coated wells with soluble MUC-1 was equally effective at blocking adhesion of GZHi to ICAM-1.

These results show that MUC-1 can selectively bind to the immunoglobulin superfamily member, ICAM-1. A possible binding site on MUC-1 for ICAM-1 is suggested by the inhibition of the interaction by B27.29. This antibody recognizes a limited sequence of the MUC-1 core peptide tandem repeat which encompasses that recognized by SM3. Reddish, et al. Tumor Marker Onc 7:19 (1992). This implicates the core peptide sequence of the tandem repeat as the binding site for ICAM-1.

### Example 5

A molecule with considerable sequence homology to ICAM-1 is ICAM-3. This adhesion molecule shows a unique pattern of expression and is found typically only in the new vasculature of solid tumors and in certain vascular hyperplasias. Patey, et al. Am. J. Pathol. 148:465-472 (1996).

Using the procedures described in Example 4, the ability of MUC-1 to bind to ICAM-3 is assessed. Once it has been determined that ICAM-3 binds to MUC-1, various treatment regimens can be developed based on the ICAM-3/MUC-1 binding.

Using the procedures described in Example 4, the ability of MUC-1 to bind to other adhesion molecules is assessed. Once adhesion molecules have been identified that bind to MUC-1, various treatment regimens can be developed.

### Example 6

One example of an autoimmune disease is myasthenia gravis. In this disease a patient produces antibodies to the acetylcholine receptor at the neuromuscular junction. This production of antibodies is dependant upon the active participation of CD+helper T cells. These T cells are activated by antigen presenting cells that express ICAM-1 which interacts with T cell LFA-1 forming an adhesion between APC and T cells. Blocking of this interaction will prevent T cell activation and the subsequent T cell help needed for the disease manifesting antibody response. Patients with symptoms of myasthenia gravis are treated by the systemic administration of a pharmaceutically acceptable amount of MUC-1, a MUC-1 derivative or a MUC-1 carbohydrate derivative, thereby down regulating the production od CD4 T cell helpers which will then turn off the production of the specific antibody producing B cells that are dependant upon T cell help. Antibody previously produced will clear from the circulation in the normal time course for IgG half-life clearance leading to a relief of symptoms of the crippling symptoms of this disease.

### Example 7

In the case of inflammatory arthritis a CD4+T cell population is known to invade the synovium of effected joints and to produce the pro-inflammatory cytokines TNF and interferon gamma. These cytokines then induce resident synovial cells to produce collagenase and other hydrolytic enzymes that degrade of collagen and destroy tendons cartilage and ligaments. The activation of these CD4 T cells requires ICAM-1 interaction with antigen presenting cells. This interaction could be blocked by the intrasynovial (or systemic) administration of a pharmaceutically effective amount of MUC-1, a MUC-1 derivative or a MUC-1 carbohydrate derivative, thereby blocking the T cell activation and subsequent production of the pro-inflammatory cytokine mediators of this crippling disease.

### Example 8

This example demonstrates the ability of synthetic peptides, having multiple tandem repeats of the MUC-1 core, to inhibit T cell proliferation.

Mucins:

MUC-1 was purified from ascites fluid obtained from ovarian cancer patients. 2M sodium acetate at pH 5 was added to the ascites fluids and centrifuged for 30 minutes at 20 krpm. After filtration through a 0.45 micron cellulose acetate filter, the solution was mixed with B27.29 Mab (Reddish et al., J. Tumor Marker Oncol. 7:19-27 (1992)) CNBr coupled to sepharose 4B overnight, followed by washing with 1M NaCl/PBS. The affinity bound MUC-1 mucin was eluted with 50 mM diethanolamine (Fisher purified) in 150 mM NaCl at pH11. The eluant was neutralized with 2M sodium acetate at pH 5. The affinity purified material was dialyzed against PBS and then sterile filtered with Nalgene 0.2 micron cellulose acetate syringe filter. The affinity purified MUC-1 mucin was quantitated by using Truquant BR RIA assay (Biomira Diagnostics Inc., Roxdale, ON, Canada). For the calculation of amount of MUC-1 mucin, the conversion formula 1 BR unit as approximately 50 ng of MUC-1 mucin, was used.

Synthetic MUC-1 derivatives contained 1, 3, 4, 5 or 6 tandem repeats of the MUC-1 core and were approximately 16, 60, 80, 100 and 120 amino acids in length. The 16-mer contained the sequence GVTSAPDTRPAPGSTA. The other derivatives contained tandem repeats of the sequence TAPPAHGVTSAPDTRPAPGS.

Ovine submaxillary mucin (OSM) was employed as a control.

T Cell Cultures:

Enriched T cell populations were purified from buffy coats obtained from normal red cross donors using nylon wool columns by previously reported procedures. See, e.g., Agrawal et al., J. Immunol. 157:2089-95 (1996) and Agrawal et al., J. Immunol. 157:3229-34 (1996). For the allo MLR, mitomycin C treated allogeneic PBLs were cocultured with purified T cells in the presence or absence of affinity purified MUC-1 mucin or control OSM. In most of the experiments, the T cells were cultured 6-7 days in AIM V medium in the absence or presence of MUC-1, MUC-1 derivative or OSM at the indicated concentration. After this time, the T cells were harvested, washed and cultured as indicated.

Proliferation Assay:

Purified T cells (10⁶/ml) were cultured in AIM V medium with allo PBLs in the absence or presence of MUC-1, MUC-1 derivative or OSM 10 ug/ml for 6-7 days. T cells were harvested and plated in 96 well flat bottom plates at 10⁵/well with allo PBLs (10⁵/well), in the presence or absence of affinity purified MUC-1, MUC-1 derivative or OSM. Control cultures were treated with either 50 U/ml IL-2 or 1 ug/ml anti-CD28 Mab. After 4 days of culture, ³H-thymidine (1 uCi/well) was added. The cells were harvested on the fifth day and ³H-thymidine incorporation was measured by liquid scintillation.

Results

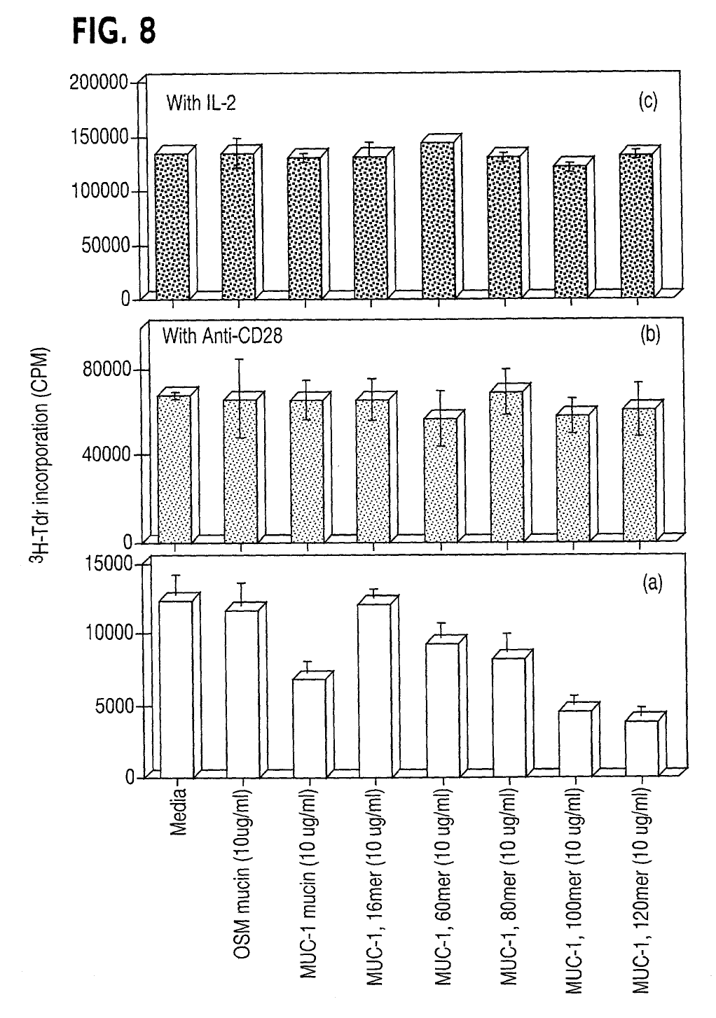

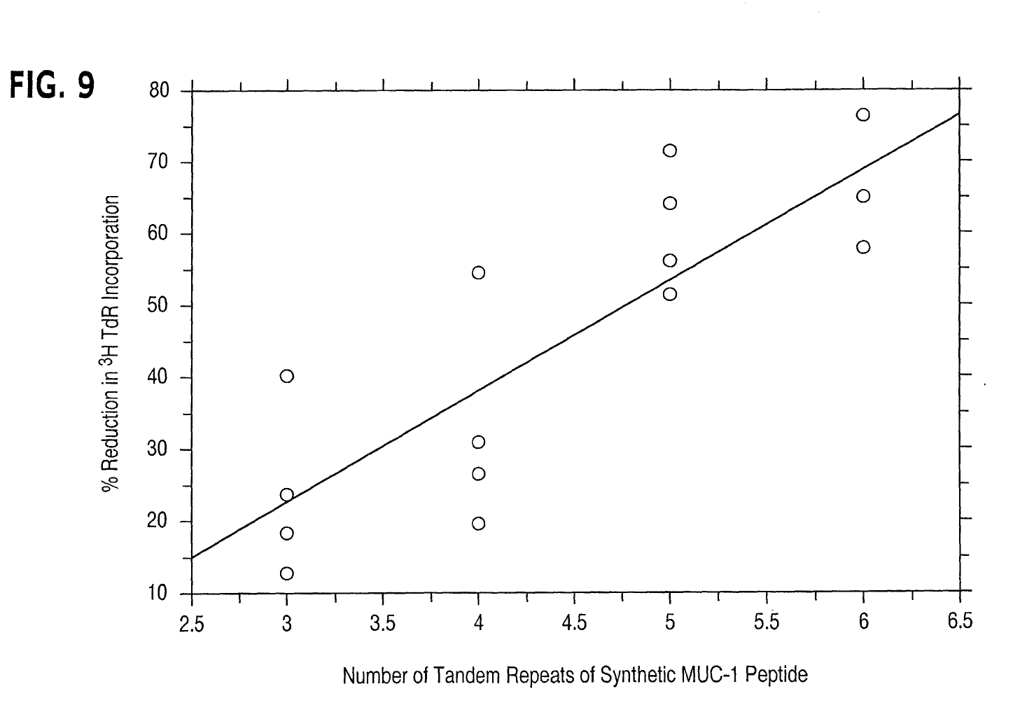

As seen in FIG. 8, synthetic peptides containing 3-6 tandem repeats of the MUC-1 core significantly reduced the level of T cell proliferation relative to control. This effect was not observed with a peptide containing a single repeat. Moreover, this effect was reversed by treatment with IL-2 or CD28 Mab. As seen in FIG. 9, inhibition of T cell proliferation was directly proportional to the number of MUC-1 core repeats present. Table 1 demonstrates the statistical significance of these data as compared to the medium control.

TABLE 1

| Sample | p |
|---|---|
| 3 repeats | =0.0009 |
| 4 repeats | =0.0007 |
| 5 repeats | <0.0001 |
| 6 repeats | <0.0001 |

Table 2 demonstrates the statistical significance of these data compared to the OSM control.

TABLE 2

| Sample | p |
|---|---|
| 3 repeats | =0.036 |
| 4 repeats | =0.003 |
| 5 repeats | <0.0001 |
| 6 repeats | <0.0001 |

### Example 9

This example illustrates the direct correlation of number of tandem repeats with the inhibit T cell proliferation. The methods used are described in Example 8.

Synthetic peptides, as described in Example 8, corresponding to 3, 4, 5 or 6 MUC-1 repeats, were added at 10 ug/ml to allo cultures and T cell proliferation was measured at 14 days from the start of culture. Because the same amount of each peptide was present in each well, all samples contained the same total number of individual repeats. Thus, the only difference was the number of repeats tandemly joined in the peptide. In other words, any effect observed is a result of the oligomeric nature of the repeats, not the absolute number of them.

As seen in FIG. 9, the number of tandemly joined repeats directly correlated with inhibition of T cell proliferation. The percent reduction in T cell proliferation was directly proportional to the number of tandem repeats of MUC-1 added to the culture (R=0.85, p<0.0001).

We claim:

1. A method of treatment, comprising administering a pharmaceutically effective amount of MUC-1, a MUC-1 derivative or a MUC-1 carbohydrate derivative to a patient in need of said treatment.

2. The method of claim 1, wherein said patient is suffering from an autoimmune disorder selected from the group consisting of inflammatory arthritis, rheumatoid arthritis, psoriasis, allergies, allergic contact dermatitis, and ankylosing spondylitis.

3. The method of claim 1, wherein said patient is suffering from an autoimmune disorder selected from the group consisting of myasthenia gravis, systemic lupus erythematosus, polyarteritis nodosa, Goodpastures syndrome, isopathic thrombocytopenic purpura, autoimmune hemolytic anemia, Grave's disease, rheumatic fever, pernicious anemia, insulin-resistant diabetes mellitus, bullous pemphigold, pemphigus vulgaris, viral myocarditis (Cocksakie B virus response), autoimmune thyroiditis (Hashimoto's disease), male infertility (autoimmune), sarcoidosis, allergic encephalomyelitis, multiple sclerosis, Sjorgens disease, Reiter's disease, Celiac disease, sympathetic ophthalmia, and primary biliary cirrhosis.

4. The method of claim 1, wherein said patient is suffering from organ transplant rejection or graft versus host disease.

5. The method of claim 1, wherein said MUC-1 derivative comprises more than one MUC-1 core repeat.

6. The method of claim 1, wherein said MUC-1 derivative comprises from about 2 to about 100 MUC-1 core repeats.

7. The method of claim 6, wherein said MUC-1 derivative comprises from about 2 to about 20 MUC-1 core repeats.

8. The method of claim 7, wherein said MUC-1 derivative comprises from about 3 to about 6 MUC-1 core repeats.

9. The method of claim 5, wherein said core repeats are arranged tandemly.

10. The method of claim 5, wherein said MUC-1 derivative is glycosylated.

11. A pharmaceutical composition comprising a pharmaceutically effective amount of MUC-1, a MUC-1 derivative or a MUC-1 carbohydrate derivative, in combination with a pharmaceutically effective vehicle.

12. The composition of claim 11, wherein said MUC-1 derivative comprises more than one MUC-1 core repeat.

13. The composition of claim 12, wherein said MUC-1 derivative comprises from about 2 to about 100 MUC-1 core repeats.

14. The composition of claim 13, wherein said MUC-1 derivative comprises from about 2 to about 20 MUC-1 core repeats.

15. The composition of claim 14, wherein said MUC-1 derivative comprises from about 3 to about 6 MUC-1 core repeats.

16. The composition of claim 12, wherein said core repeats are arranged tandemly.

17. The method of claim 12, wherein said MUC-1 derivative is glycosylated.

18. The method of claim 11, wherein said pharmaceutically effective amount is an amount effective for treating an autoimmune disorder or an inflammatory disorder.

19. The method of claim 11, wherein said pharmaceutically effective amount is an amount effective for treating organ transplant rejection or graft versus host disease.
